(19) United States
(12) Patent Application Publication
(10) Pub. No.: US 2024/0350115 A1
(43) Pub. Date: Oct. 24, 2024

(54) Method and apparatus for backscattering imaging vascular activity at a microscopic scale

(71) Applicants: ICONEUS, Antony (FR); Institut National de la Santé et de la Recherche Médicale, Paris (FR); CENTRE NATIONAL DE LA RECHERCHE SCIENTIFIQUE, Paris (FR); Ecole Supérieure de Physique et de Chimie Industrielles de la Ville de Paris, Paris (FR)

(72) Inventors: Noémi RENAUDIN, Paris (FR); Mickaël TANTER, Bagneux (FR); Charlie DEMENÉ, Cambridge (GB); Bruno-Felix OSMANSKI, Sartrouville (FR)

(21) Appl. No.: 18/304,471

(22) Filed: Apr. 21, 2023

(51) Int. Cl. A61B 8/08; A61B 8/00; G06T 7/11; G06T 7/246; G06T 7/73; G06T 11/00

(52) U.S. Cl. CPC A61B 8/0891; A61B 8/4494; A61B 8/481; A61B 8/5207; G06T 7/11; G06T 7/246; G06T 7/73; G06T 11/00; G06T 2207/10056; G06T 2207/10132; G06T 2207/30104; G06T 2207/30241

(57) ABSTRACT

Method for imaging vascular activity at a microscopic scale in at least one area of a vascular network of an organ, of a human or animal, the method including:
(a) transmitting a series of successive incident ultrasonic waves in the at least one area by an array of ultrasonic transducers, the array of ultrasonic transducers extending along at least one direction and the incident ultrasonic waves being propagated in a direction perpendicular to the array of transducers;
(b) acquiring a set of raw data from backscattered ultrasonic waves by said array of transducers;
(c) generating a series of successive ultrasound images from said raw data;
(d) detecting at least one isolated ultrasound contrast agent in the ultrasound images;
(e) localizing the position of said at least one isolated ultrasound contrast agent with a precision inferior to the wavelength of the waves;
(f) generating an at least 2D backscattering amplitude image by attributing for each pixel a value representative of the measured backscattering amplitude of at least one isolated ultrasound contrast agent detected in said pixel.

### TECHNICAL FIELD

The present disclosure concerns methods and apparatuses for backscattering imaging vascular activity at a microscopic scale in a human or animal.

### BACKGROUND ART

One type of imaging of high interest, particularly regarding spatial resolution and penetration depth, is Ultrasound Localization Microscopy (ULM), which has been described in particular by Errico et al. [Errico, C. et al. Ultrafast ultrasound localization microscopy for deep super-resolution vascular imaging. Nature 527, 499-+ (2015)] and Demene et al. [Demene, C. et al. Transcranial ultrafast ultrasound localization microscopy of brain vasculature in patients. Nat. Biomed. Eng 5, 219-228 (2021)]. By imaging isolated intravenously injected microbubbles (MB) circulating in blood vessels at ultrafast frame rates and localizing the center of their individual point spread function with a sub-resolution precision, it enables to map the microvascularization of organs with a resolution of tens of microns and blood flow dynamic velocities.

However, in conventional ULM images which are based on microbubbles count, the signal is driven by blood flow. In other words, the bigger the blood flow, the more MBs are detected during the acquisition time. Thus, many small vessels are not visible in the conventional ULM images due to the noise level in areas dimply exploring by the microbubbles.

Furthermore, ULM is generally implemented in two-dimension and provides 2D images, as both the linear transducers arrays and driving electronics are far more common and less expensive than matrix arrays, raw-column arrays, and multiplexed electronics required to perform 3D ultrasound localization microscopy. Thus, velocity quantification is limited only to in-plane velocity which underestimates velocity measurement of microbubbles' flow that are not aligned in the ULM imaging plane. This lack of information in the direction orthogonal to the imaging plane leads to an inaccurate velocity determination.

Therefore, there exists a need for an improved method for imaging vascular activity at a microscopic scale, less sensitive to noise, allowing the detection of small vessels, while improving the accuracy of velocity quantification using 2D ULM.

### SUMMARY

To this end, the present disclosure proposes a method for imaging vascular activity at a microscopic scale in at least one area of a vascular network of an organ, of a human or animal, the method including:
(a) transmitting a series of successive incident ultrasonic waves in the at least one area by an array of ultrasonic transducers, the array of ultrasonic transducers extending along at least one direction and the incident ultrasonic waves being propagated in a direction perpendicular to the array of transducers;
(b) acquiring a set of raw data from backscattered ultrasonic waves by said array of transducers;
(c) generating a series of successive ultrasound images from said raw data;
(d) detecting at least one isolated ultrasound contrast agent in the ultrasound images;
(e) localizing the position of said at least one isolated ultrasound contrast agent with a precision inferior to the wavelength of the waves;
(f) generating an at least 2D backscattering amplitude image by attributing for each pixel a value representative of the measured backscattering amplitude of at least one isolated ultrasound contrast agent detected in said pixel.

The proposed method advantageously allows, depending on the embodiment, to obtain backscattering ULM images with a higher sensitivity compared to conventional ULM images. During the processing to generate ULM images, microbubbles are detected as brightest local maxima. The value of this maxima on the BMode images are extracted and associated with each localized microbubble detection. This value is referred as the backscattering amplitude. Then, instead of attributing to each ULM map's pixel the number of microbubbles detected in the pixel during the whole acquisition, a mean value of the backscattering amplitude of the MBs detected in this pixel. Owing to the measurement of the backscattering amplitude of each individual microbubble circulating in a vessel, it is possible to retrieve the missing information about the out-of-plane motion of microbubbles. Thus, backscattering ULM images allow a physically relevant 3D rendering perception in the vascular map. information from the individual ULM images may be gathered. The gathering of information from the individual ULM images may reduce the acquisition period of the individual ULM images, while maintaining a high spatial resolution.

The following features, can be optionally implemented, separately or in combination one with the others:

In at least one embodiment, the value computed in step (f) may correspond to an average of the measured backscattering amplitude of all isolated ultrasound contrast agents detected in said pixel.

In at least one embodiment, the value computed in step (f) may correspond to a function of the backscattering amplitude of the at least one isolated ultrasound contrast agents detected in said pixel.

In one embodiment, the method may further comprise a tracking step wherein at least one isolated ultrasound contrast agent detected is tracked across several ultrasound images generated in step (c) to determine its trajectory and its speed.

In some embodiments, the array of ultrasonic transducers may be a 1D array extending along one direction X and the incident ultrasonic waves being propagated in a direction Z perpendicular to the array of transducers, said image generated in step (f) is a 2D backscattering amplitude image.

In the embodiment wherein the array of ultrasonic transducers is a 1D array, the steps (a) to (f) may be repeated for a plurality of parallel ultrasound imaging planes XZ spaced from each other within the width of the ultrasound beam along the direction Y to generate a plurality of 2D parallel backscattering amplitude images.

In some embodiments, the method may further comprise a computation speed step for the at least one tracked isolated ultrasound agent at a given position (x, z) comprising the following steps:
(i) generating a spatial variation of the backscattering amplitude for said at least one tracked isolated contrast agent as a function of the position y of the imaging plane along the direction Y within the width of the ultrasound beam;
(ii) computing the position y of said at least one tracked isolated contrast agent by fitting a function on the spatial variation of the measured backscattering amplitude, the position y of the at least one isolated contrast agent being the position of the maximum of the spatial variation of backscattering amplitudes;
(iii) computing the velocity components Vₓ, V_{y} and V_{z} along respectively the three directions X, Y, Z for said at least one tracked isolated ultrasound contrast agent.

In one or more embodiments, the method may further comprise a computation speed step for each pixel at a given position (x, z) comprising the following steps:
(i) generating a spatial variation of the backscattering amplitude for each pixel as a function of the position y of the imaging planes along the direction Y within the width of the ultrasound beam;
(ii) computing the position y of each pixel by fitting a function on the spatial variation of the measured backscattering amplitude, the position y of each pixel being the position of the maximum of the variation of backscattering amplitudes;
(iii) computing three velocity components Vₓ, V_{y} and V_{z} along respectively the three directions X, Y, Z for each pixel

In one or more embodiments, the fitting function may be a Gaussian function:

$$a \times {e}^{- ( \frac{y - {y}_{0}}{w} ) 2} ,$$

a being the backscattering amplitude, w being the width of the ultrasound beam along the direction Y, and y₀ being the position of the maximum of the variation of backscattering amplitudes.

In one or more embodiments, the width w of the ultrasound beam along the direction Y may be an experimental value.

In one or more embodiments, the width w of the ultrasound beam along the direction Y may be a simulated value.

In one or more embodiments, the plurality of parallel ultrasound imaging planes may be obtained by moving the 1D array of ultrasonic transducers along a direction Y perpendicular to the ultrasound imaging plane (x, z) within the width (w) of the ultrasound beam.

In one or more embodiments, the method may further comprise a correction step of the positioning of the 1D array of ultrasonic transducers by comparing a first backscattering amplitude image and a second backscattering so as the imaging plane associated to the first backscattering amplitude image coincides with the imaging plane associated to the second backscattering image.

In some embodiments, the method may further comprise a step of generating an at least 2D ultrasound localization microscopy (ULM) image of the at least one area is generated by attributing for each pixel a number of ultrasound contrast agents detected in said pixel.

In some embodiments, the method may further comprise a step of generating an at least 2D ultrasound localization microscopy (ULM) image of the at least one area is generated by attributing for each pixel a number of ultrasound contrast agents detected in said pixel.

In one embodiment, the method may further comprise a quantification step of the corrected velocity for at least one vessel from said 2D backscattering amplitude image, said quantification step comprising:
(i) selecting said at least one blood vessel in the at least one backscattering amplitude image;
(ii) computing the velocity V_{xz} of the ultrasound contrast agents belonging to said selected blood vessel;
(ii) obtaining the backscattering amplitude profile along a centerline of said selected blood vessel;
(iii) computing the angle θ between the backscattering amplitude profile along the selected blood vessel centerline and its projection on the ultrasound imaging plane;
(iv) computing a corrected velocity for each ultrasound contrast agents belonging to said selected vessel using said computed angle: V_{corr}=V_{xz}/cos θ.

In some embodiments, the step of generating of a series of successive 2D ultrasound images from said raw data may comprise a step of filtration to discriminate the ultrasound signal of the individual ultrasound contrast agents from a tissue signal.

In one or more embodiments, at least one vascular parameter may be extracted from the measured backscattering amplitudes, said one vascular parameter being chosen in the group comprising: blood flow, blood velocity, blood volume, blood pressure and any combination thereof.

Computing a dynamics parameter among blood flow, blood velocity, blood volume, blood pressure, and any combination thereof may allow to derive a behavior of the at least one region of the vascular network, enabling to quantify the activity of the at least one area without requiring a large amount of information from different types.

In one or more embodiments, the method may further comprise an automatic segmentation step of vessels in at least one area of a vascular network from the at least 2D backscattering amplitude image and wherein at least one dimensional parameter of vessels is quantified from the segmentation step. For instance, the diameter may be obtained by different segmentation techniques applied to the backscattering amplitude images. In its simplest form, it could be defined as a width of the vessel at a threshold set by the Half Maximum of a rest profile.

The present disclosure also concerns an apparatus for imaging vascular activity at a microscopic scale in at least one area of a vascular network of an organ, of a human or animal, said apparatus including:
(a) an ultrasound probe having an array of ultrasonic transducers extending along at least one direction, said ultrasound probe configured to transmit incident ultrasound waves in a direction of propagation perpendicular to the array of transducers and acquire a set of raw data;
(b) a computing module configured to:
   generate a series of successive ultrasound images from said raw data;
   detect at least one isolated ultrasound contrast agent in the ultrasound images;
   localize the position of said at least one isolated ultrasound contrast agent with a precision inferior to the wavelength of the waves;
   track said at least one isolated ultrasound contrast agent to determine its trajectory;
   generate an at least 2D backscattering amplitude image by attributing for each pixel a value representative of the measured backscattering amplitude of at least one isolated ultrasound contrast agent detected in said pixel.

In embodiments of the apparatus, one may use the following features, alone or in combination:

In one or more embodiments, the array of ultrasonic transducers may be 1D array of ultrasonic transducers extending along one direction X to generate 2D backscattering amplitude images.

In some embodiments, the probe may further comprise a motor configured to move the array of ultrasonic transducers along a direction perpendicular to the ultrasound imaging plane within the width (w) of the ultrasound beam to generate a plurality of parallel ultrasound imaging planes.

In one or more embodiments, the computing module may further be configured to:
generate a spatial variation of the backscattering amplitude for said at least one tracked isolated contrast agent as a function of the position y of the imaging planes along the direction Y within the width of the ultrasound beam;
compute the position y of said at least one tracked isolated contrast agent by fitting a function on the spatial variation of the measured backscattering amplitude, the position y of the at least one isolated contrast agent being the position of the maximum of the variation of backscattering amplitudes;
compute three velocity components Vₓ, V_{y} and V_{z} along respectively the three directions X, Y, Z for said at least one tracked isolated ultrasound contrast agent.

In another aspect, it is proposed a computer software comprising instructions to implement a method for imaging vascular activity at a microscopic scale in at least one area of a vascular network of an organ, of a human or animal, when the software is executed by a processor, said method including:
(a) transmitting a series of successive incident ultrasonic waves in the at least one area by an array of ultrasonic transducers, the array of ultrasonic transducers extending along at least one direction and the incident ultrasonic waves being propagated in a direction perpendicular to the array of transducers;
(b) acquiring a set of raw data from backscattered ultrasonic waves by said array of transducers;
(c) generating a series of successive ultrasound images from said raw data;
(d) detecting at least one isolated ultrasound contrast agent in the ultrasound images;
(e) localizing the position of said at least one isolated ultrasound contrast agent with a precision inferior to the wavelength of the waves;
(f) generating an at least 2D backscattering amplitude image by attributing for each pixel a value representative of the measured backscattering amplitude of at least one isolated ultrasound contrast agent detected in said pixel.

In another aspect, it is proposed a computer-readable non-transient recording medium on which a software is registered to implement a method for imaging vascular activity at a microscopic scale in at least one area of a vascular network of an organ, of a human or animal, when the software is executed by a processor, said method including:
(a) transmitting a series of successive incident ultrasonic waves in the at least one area by an array of ultrasonic transducers, the array of ultrasonic transducers extending along at least one direction and the incident ultrasonic waves being propagated in a direction perpendicular to the array of transducers;
(b) acquiring a set of raw data from backscattered ultrasonic waves by said array of transducers;
(c) generating a series of successive ultrasound images from said raw data;
(d) detecting at least one isolated ultrasound contrast agent in the ultrasound images;
(e) localizing the position of said at least one isolated ultrasound contrast agent with a precision inferior to the wavelength of the waves;
(f) generating an at least 2D backscattering amplitude image by attributing for each pixel a value representative of the measured backscattering amplitude of at least one isolated ultrasound contrast agent detected in said pixel . . .

### BRIEF DESCRIPTION OF DRAWINGS

Other features, details and advantages will be shown in the following detailed description and on the figures, on which:

FIG. 1 is a block diagram illustrating an embodiment of an apparatus according to the present disclosure.

FIG. 2 is a block diagram illustrating an embodiment of a method of obtaining a 2D or 3D backscattering amplitude ULM image and a 2D or 3D conventional ULM image with the apparatus of FIG. 1.

FIG. 3 illustrates a possible method of obtaining a 2D or 3D backscattering amplitude ULM image and a 2D or 3D conventional ULM image with the apparatus of FIG. 1.

FIG. 4(a) illustrates schematically an ultrasound beam generated by a linear array of transducers and a microbubble or an isolated contrast agent (MB) flowing in a blood vessel crossing the imaging plane at two times t₁ and t₂.

FIG. 4(b) illustrates two BMode images of the microbubble at t₁ and t₂ along the direction Y orthogonal to the imaging plane.

FIG. 4(c) illustrates a simulated variation of the backscattered ultrasound intensity as a function of y for a fixed position (x₀, y₀).

FIG. 5(a) illustrates simulated backscattering intensity in the YZ plane at the X central position (X=0) and simulated backscattering intensity in the YZ plane at the X central position (X=0) normalized for each depth along the direction Z.

FIG. 5(b) illustrates a decrease of ultrasound intensity profile with depth along the direction Z for x=0 and y=0.

FIG. 5(c) illustrates ultrasound intensity profile along the direction Y for a given depth z and normalized for each depth.

FIG. 6 illustrates an example of a backscattering amplitude ULM image representing the vessels in a rat thalamus according to an embodiment of the method of the present disclosure.

FIG. 7(a) illustrates schematically a variation of the backscattering amplitude for a microbubble flowing through a blood vessel crossing the ultrasound beam along the direction Y.

FIG. 7(b) represents normalized backscattering amplitude as a function of time of one microbubble flowing in the blood vessel whose trajectory is shown on FIG. 6 as a white line.

FIG. 8(a) illustrates schematically a variation of the backscattering amplitude for microbubbles flowing through blood vessels parallel to the imaging plane and distributing along the direction Y.

FIG. 8(b) illustrates an example of a backscattering amplitude image representing the blood vessels in the rat cortex.

FIG. 9(a) illustrates schematically variation of the ultrasound intensity for five imaging planes spaced from each other in the direction Y and three microbubbles at a given fixed position (x, z) flowing in a blood vessel crossing the ultrasound beam along the direction Y.

FIG. 9(b) illustrates schematically the backscattering amplitude for the three microbubbles measured in the five imaging planes.

FIG. 10 represents five backscattering amplitude images of five coronal planes in the rat thalamus spaced by 100 μm from each other in the elevation direction Y.

FIG. 11 represents five backscattering amplitude normalized values measured for the pixel M showing on FIG. 10 and a gaussian function fitted on the five points.

FIG. 12(a) represents four selected blood vessels on the backscattering amplitude image.

FIG. 12(b) represents schematically a geometric construction to determine the angle θ between the selected blood vessel numbered 2 and its projection on the imaging plane.

### DESCRIPTION OF EMBODIMENTS

In the Figures, the same references denote identical or similar elements.

The present disclosure proposes a method and apparatus for backscattering amplitude imaging vascular activity in at least one area of an organ of a human or animal. This backscattering amplitude imaging uses backscattering amplitudes in Ultrasound Localization Microscopy (ULM) to provide 2D or 3 D ultrasound backscattering amplitude images with a higher sensitivity compared to conventional ultrasound localization microscopy images. It is possible to visualize small vessels that are not visible on conventional ULM images due to an intrinsic lower spatial noise. As backscattering amplitudes vary along the direction orthogonal to the 2D imaging plane of the ultrasound probe, it is also possible to render a 3D perception in a 2D vascular image.

In addition, in embodiments wherein the ultrasound probe includes a linear array adapted to generate a 2D image of an area of interest, the backscattering amplitude images enable to retrieve the relative distance of the ultrasound contrast agents to the 2D imaging plane and the 3D localization, allowing a more accurate velocity quantification by using the missing information about the out-of-plane motion of microbubbles. In other words, the present disclosure provides a method and apparatus to compute 3D flow and velocity from 2D ULM images obtained with a 1D ultrasound probe.

2D or 3D conventional ULM images may be obtained based on methods already known in the art and explained in the above articles of Errico et al., 2015 and Demene et al., 2021.

The backscattering amplitude images are generated from the same set of raw date that are used to generate the conventional ULM images.

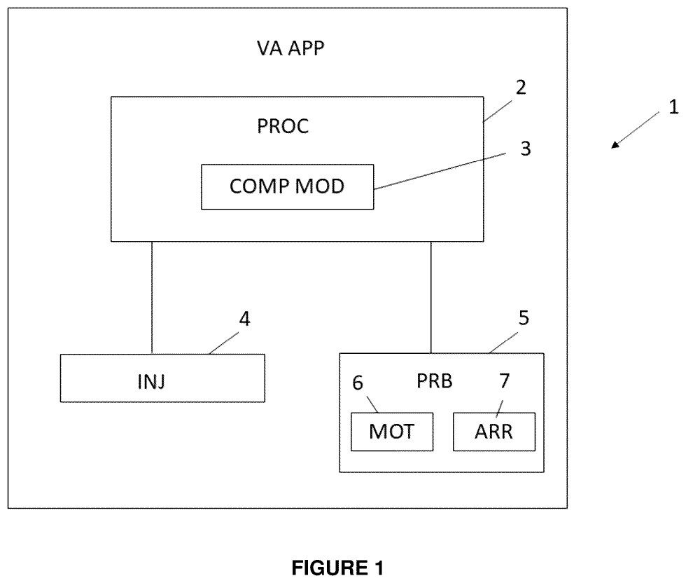

An example of apparatus 1 (VA APP) for imaging vascular activity usable in performing the method according to the present disclosure, is shown on FIG. 1.

The apparatus 1 may include a processor 2 (PROC), for instance a specialized signal processing device controlled by a computer or a group of computers, possibly a group of computers including servers.

The processor 2 may include a computing module 3 (COMP), the operation of which will be explained later.

The processor 2 may control an ultrasound contrast agent injection device 4 (INJ).

The ultrasound contrast agent injection device 4 may comprise ultrasound contrast agents. The ultrasound contrast agents may be microbubbles, as for instance described by Dayton et al., [Dayton, P A et al. Molecular ultrasound imaging using microbubble contrast agent. Frontiers in Bioscience 12, 5124-5142 (2007)], or equivalent ultrasound contrast agents. In some embodiments, the ultrasound contrast agents may be based on SonoVue®.

The ultrasound agent injection device 4 may be a push syringe in the example considered here.

The ultrasound agent injection device 4 may comprise a magnet in order to mix a solution comprising the ultrasound contrast agents.

The processor 2 may control an ultrasound probe 5 (PRB).

The ultrasound probe 5 may include an array 7 (ARR) of ultrasonic transducers. The array may be a linear array adapted to generate a 2D backscattering amplitude image of a sliced of the area to be imaged, or a 2D array adapted to generate a 3D backscattering amplitude image of the area. When the array is a 2D array, it may be a sparse matrix of transducers, as known in the art.

The process of generating a backscattering amplitude image would be similar for the 1D array and the 2D array. The following detailed description is done for the case of a linear array.

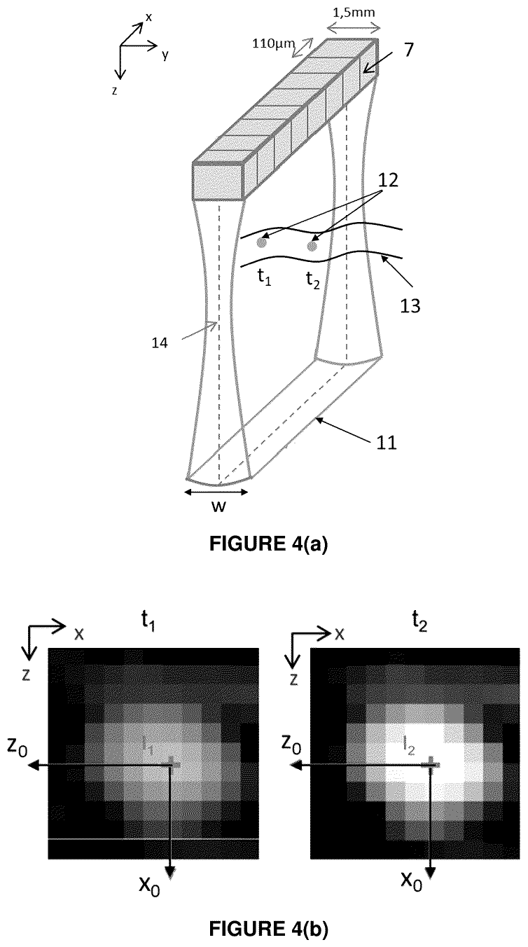

FIG. 4(a) represent an example of a linear array of ultrasonic transducers aligned along an axis X and adapted to transmit ultrasound waves along an axis Z perpendicular to the axis X. Thus, the imaging plane 14 of the ultrasound probe extends in a XZ plane. A typical ultrasound probe may comprise a few hundreds of transducers adapted to image a plurality of lines of the region, in the direction of the depth Z from the transducers, so that the apparatus generates 2D images having pixels. In addition, each transducer has a geometrical focusing in the direction orthogonal to the imaging XZ plane along the axis Y, called the direction of elevation or the direction out-of-plane. For all examples illustrated in the present disclosure, the 1D array ultrasonic probe comprises 128 ultrasonic transducers, with a 110 μm.

The transducers may be adapted to transmit and receive ultrasound waves having a central frequency comprised for instance between 0.5 and 100 MHz, for instance between 1 and 20 MHz. One example of usable central frequency is 15 MHz.

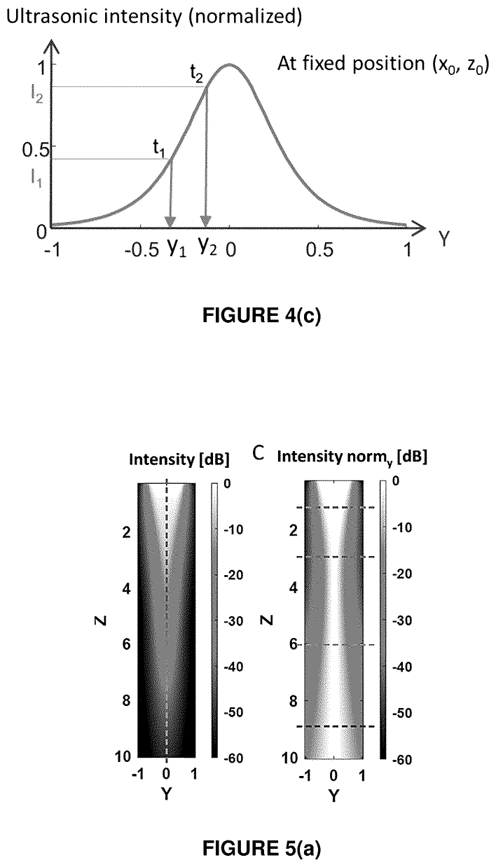

The FIGS. 5(a)-5(c) show the ultrasound beam pressure field and acoustic intensity obtained by simulation for the linear array of FIG. 4(a). The spatial ultrasound field transmitted by the ultrasonic probe is simulated using Field II software. The simulation parameters are given in Table 1.

TABLE 1

| Ultrasound probe parameters | | | Simulation parameters | | |
|---|---|---|---|---|---|
| Frequency | 15.625 | MHz | Speed of sound | 1540 | m/s |
| Nb piezo elements | 128 | | Attenuation | 134.24 Np/m (value for brain | |
| | | | | white matter at 15.625 MHz | |
| | | | | ©2010-2022 IT’IS | |
| | | | | Foundation) | |
| Dimension piezo elements: | | | Spatial sampling: | | |
| X | 108 | μm | X | 100 | μm |
| Y | 1.5 | mm | Y | 10 | μm |
| | | | Z | 100 | μm |
| Inter element distance (kerf) | 2 | μm | Sampling frequency | 300 | MHz |

| | | | | |
|---|---|---|---|---|
| Elevation focus | 8 | mm | Input waveform | 2 periods of sinusoid |

The 3D pressure field is simulated in space and time after emission of a plane wave. In each point of space (x, y, z) is therefore recorded a pressure signal p (t).

FIG. 5(a) shows a map of ultrasound intensity obtained in the YZ plane at the x central position (x=0) and a map of ultrasound intensity in the YZ plane at the x central position normalized for each depth z.

FIG. 5(b) shows that the ultrasound intensity decreases exponentially with depth z because of ultrasonic attenuation along the axis Z.

FIG. 5(c) shows that the ultrasound intensity profile along the axis Y for four given depth z (1 mm, 3 mm, 6 mm and 9 mm), normalized for each depts. The simulation shows that the ultrasound intensity varies in 3D space. In particular, the simulation shows that in the direction orthogonal to the imaging plane, the ultrasound beam has a certain width w that varies along the axis Z. Thus, for a fixed position (x₀, z₀) in the imaging plane 14, the ultrasound intensity is maximal in the middle and decreases from either side as illustrated in FIG. 5(c).

In certain embodiments, the probe 5 may further include a motorization 6 (MOT) adapted to move the array 7 along the three axis XYZ.

According to an embodiment, the motorization 6 is configured to move the 1D array of ultrasonic transducers along the direction Y perpendicular to the ultrasound imaging plane XZ within the width w of the ultrasound beam to generate a plurality parallel ultrasound imaging planes along the axis Y. Thus, the same area may be imaged and the same blood vessels belonging to the said area are present on the 2D ultrasound images.

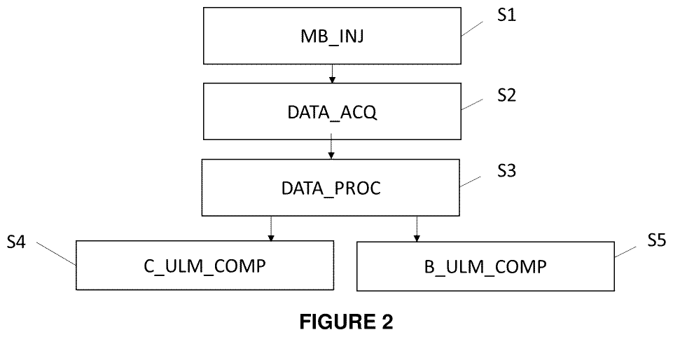

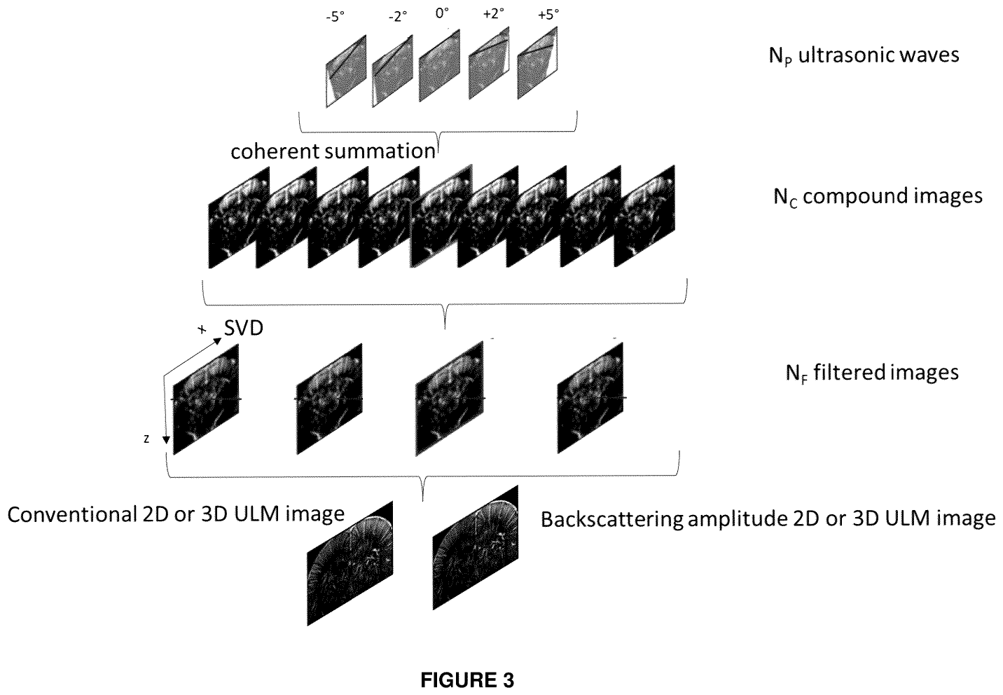

An example of method of convention Ultrasound Localization Microscopy imaging, already known in the art and explained for instance in the above article of Demene et al., 2021, will now be explained with regards to FIG. 2 and FIG. 3.

The ultrasound contrast agent injection device 4 may be controlled by the processor 2 to inject S1 (MB_INJ) intravenously microbubbles or ultrasound contrast agents to a region of the vascular network.

The ultrasound contrast agent injection may be a bolus injection with a maximum injection volume of 40 mL/kg for rats (corresponding to 12 mL for a 300 g rat and approximately 3 mL for a 70 g mice) and a typical 6 mL injection volume for rats and 2 mL injection volume for mice. For a human, a maximum volume of 10 mL may be injected. For instance, two bolus of 2.4 mL may be injected in the human.

The ultrasound contrast agent injection may be a continuous injection. For instance, a flow rate of the continuous injection may be comprised between 3 mL/h/kg and 60 mL/h/kg, and preferably approximatively 10 mL/h/kg (or typically 3.5 mL/h for a 300 g rat and 1.0 mL/h for a 70 g mice). Continuous injections enable a stable number of microbubbles for more than 20 minutes with approximately 30 microbubbles per ultrasound frame which correspond to a compound image.

The array 7 of transducers may be controlled by the processor 2 to acquire S2 (DATA_ACQ) compound images of the area of the vascular network during the ultrasound contrast agent injection or after having injected the ultrasound contrast agent.

The computing module 3 may be configured to process S3 (DATA_PROC) the acquired compound images of the region to obtain filtered images of the region.

The computing module 3 may be configured to compute S4 (C_ULM_COMP) 2D or 3D conventional ULM images of the area of the vascular network based on the filtered images of the areas.

The computing module 3 may be further configured to compute S5 (B_ULM_COMP) 2D or 3D backscattering amplitude images of the area of the vascular network based on the filtered images of the areas.

The 1D array 7 of ultrasonic transducers may be controlled by the processor 2 to acquire S2 compound images by transmitting ultrasonic waves in the area of the vascular network to be imaged and by receiving the resulting backscattered ultrasonic waves, at a rate of for instance 5 kHz (Pulse Repetition Frequency PRF), i.e. every 0.2 ms. More generally, the Pulse Repetition Frequency PRF may be over 500 Hz. The received signals may be registered as a set of raw data for each transmitted ultrasonic wave. The successive transmitted waves may have propagation directions which are inclined of varying successive angles with respect to the direction Z of the depth in the area to be imaged.

For each image of the area, a number Nₚ of ultrasonic waves may be successively transmitted with different angles and the Nₚ sets of raw data may be coherently added to synthesize said image of the region, which is thus a compound image.

For instance, in the example illustrated in FIG. 3, Nₚ may be 5 with angles taking the values −5 deg, −2 deg, 0 deg, +2 deg, +5 deg. In the case of Nₚ=5 and PRF=5 kHz, the rate of the compound images of the region (framerate) is thus 1 kHz. Nₚ may be different than 5, in which case the framerate of compound images is different. For instance, Nₚ=11 may be used. The array 7 of transducers may be placed on a skull of a human or animal for transcranial experiments, or directly on a brain of the human or animal for experiments involving a craniotomy. The ultrasonic waves may be diverging waves, planar waves, or unfocused waves.

Based on the successive compound images of the area of the vascular network, filtered images may then be computed S3 by the computing module 3. In the example of FIG. 3, N_{c} successive compound images are used for each filtered image. The N_{c} successive compound images may be temporally continuous or may partially overlap in the temporal dimension. For instance, N_{c} may be comprised between 10 and 1000, for instance between 200 and 600. One example of usable number of successive compound images for one filtered image is 400. Time positions may be associated to the filtered images. A time position for a given filtered image may correspond to an average of acquisition times of the N_{c} compound images forming the filtered image.

In an embodiment wherein the array of ultrasonic transducers is a linear array as illustrated in FIG. 4(a), a spatial variation of the measured backscattering amplitude for at least one isolated ultrasound agent at a given position (x, z) may be generated as a function of the position y of the imaging plane along the direction Y within the width of the ultrasound beam to determine the position y of the at least one isolated ultrasound agent. In this embodiment, the processor 2 may be configured to move the 1D array along the direction Y to generate a plurality of parallel ultrasound imaging planes spaced from each other within the width of the ultrasound beam in the direction Y. For instance, the probe is moved to generate 5 imaging planes along the Y axis with a 100 μm step between each of the 5 planes. For the first imaging plane, a block of 400 compound images is acquired before moving to the next imaging plane to generate the block of 400 compound images. At the end of 5th imaging plane, the 1 array of ultrasonic transducers is moved back to the first imaging plane to start the measurement of the second block of 400 compound images. Thus, and according to an example, 525 blocks of 400 compound images are acquires for each imaging plane. A 150 ms pause may be required between two imaging planes to let enough time for the motor to move the 1D array of ultrasound transducers. The 525 blocks of 400 compound images may be used to create a backscattering amplitude 2D image and a conventional 2D ULM image.

The filtered images may be computed for instance by a Singular Value Decomposition (SVD). More specifically, a SVD spatiotemporal clutter filter, as for instance described by Demene et al. [Demene, C. et al. Spatiotemporal Clutter Filtering of Ultrafast Ultrasound Data Highly Increases Doppler and fUltrasound Sensitivity. IEEE Transactions on Medical Imaging 34, 2271-2285 (2015)], may be applied. In the example of FIG. 3, each filtered image may be a singular value or a sum of singular values of the SVD applied to the N_{c} compound images. In some embodiments, an ultrasonic signature of the individual ultrasound contrast agents may be discriminated from a tissue signal, due to cardiac and breathing pulsatility, of the region by performing a speckle tracking correlation analysis of the compound images prior to applying the SVD, as described by Hingot et al. [Hingot, V. et al. Subwavelength motion-correction for ultrafast ultrasound localization microscopy. Ultrasonics 77, 17-21 (2017)]. Alternatively, owing to the SVD of the compound images, for each filtered image, the ultrasonic signature of the individual ultrasound contrast agents may be discriminated from the tissue signal of the region. By discarding some singular values of the SVD, the tissue signal may be filtered. For instance, the first 10 singular values of the SVD, mostly reflecting the tissue signal of the region, may be discarded.

Each of the filtered images may be interpolated. For instance, each of the filtered images may be interpolated by a Lanczos interpolation kernel. Each of the filtered images may be interpolated to achieve a sampling rate in the order of (α/6×ε/6), where a is a spatial pitch of the probe and & is a wavelength of the ultrasounds.

A stack of the filtered images may be filtered based on a vesselness filtering, as for instance described by Jerman et al. [Jerman, T. et al. Enhancement of Vascular Structures in 3D and 2D Angiographic Images. IEEE Transactions on Medical Imaging 35, 2107-2118 (2016)].

In the step of processing of the compound images S3, Ultrasound contrast agents may be detected in the filtered images. For instance, ultrasound contrast agents may be detected as the brightest local maxima with high correlation with a point spread function. High correlation may be defined as a correlation superior to a threshold. The threshold may be a value comprised between 0.5 and 1. For instance, the threshold may be equal to 0.7. The point spread function is an imaging response of an isolated ultrasound contrast agent, which may be modelled as a Gaussian spot of axial and lateral dimension of E. Sub-pixel maxima localization may be performed using a fast local second-order polynomial fit. A neighborhood for the fast local second-order polynomial fit may be a 5×5 pixel neighborhood. Coordinates of the localized sub-pixel maxima may be rounded to a chosen pixel size. The chosen pixel size may be inferior to the pixel size of the filtered image. For instance, the chosen pixel size may be a submultiple of the pixel size of the filtered image. The submultiple may be a multiple of 2. For instance, the submultiple may be equal to 16. For instance, the chosen pixel size may be equal to initial pixel size/8.

In the step of processing of the compound images S3, Ultrasound contrast agents may be tracked in the filtered images. A tracking of the ultrasound contrast agents may be performed using a particle tracking algorithm known in the art. Tracks may be computed based on the tracking of the ultrasound contrast agents in the filtered images. A track may correspond to positions of a tracked ultrasound contrast agent in the filtered images. Each position of a tracked ultrasound contrast agent in the filtered images may be associated with a time position corresponding to the time position of the filtered image in which the position is located. Tracks with ultrasound contrast agents detected in a predefined number of successive filtered images may be computed. The predefined number of successive filtered images may be comprised between 1 and 100. For instance, the predefined number of successive filtered images may be 10. A spatial interpolation may be computed for each track in order to obtain one ultrasound contrast agent in each pixel located on the path between two successive pixels of the track.

Successive positions of ultrasound contrast agents in a track may be used to compute velocity parameters. For example, interframe ultrasound contrast agent velocity vector components may be computed along the probe x-axis and the depth z-axis for 2D imaging.

Based on the filtered images, the conventional 2D ULM images may be computed S4 (ULM_COMP) by the computing module 3. The conventional ULM images may be computed based on the tracks, which are based on the filtered images. In the example of FIG. 3, N_{F} filtered images are used for each ULM image. Each conventional ULM image may be constructed by selecting a pixel size and by sorting each detected ultrasound contrast agent of the N_{F} filtered images within each pixel. In some embodiments, values of pixels accumulating a value of detected ultrasound contrast agents in the N_{F} filtered images inferior to a threshold are set to zero. The threshold may be comprised between 1 and 10, preferably 5.

ULM images of ultrasound contrast agent count may be computed based on the detected ultrasound contrast agents. For instance, ULM count images may be computed by counting, for each pixel, the number of ultrasound contrast agents detected in the corresponding pixel of the filtered images. In the example of FIG. 3, for each pixel, the number of ultrasound contrast agents detected in the corresponding pixel of the N_{F} filtered images may be used. For instance, the pixel size for image construction is around 13 μm.

A slow-motion drift may occur in the ULM images potentially due to recording periods superior to a period of the cardiac cycle and to a period of the breathing cycle. The slow-motion drift may also result from an anesthesia in anesthetized humans or animals. In cases where a craniotomy is performed, potential brain swelling of the brain may contribute to the slow-motion drift. A correction of the slow-motion drift may be performed via an intensity-based spatial registration. The intensity-based spatial registration may be one of a translation transformation, a translation and a rotation transformation or a more complex non rigid transformation. The intensity-based spatial registration may be performed based on a ULM image of a period inferior to the recording period. For instance, a 10 s ULM count image may be used to correct the drift occurring in the ULM images of a larger recording period. Correction of the slow-motion drift may enable to observe the dynamical event object of the present disclosure, which is due to a cause other than cardiac pulsatility and which may be slower than the cardiac pulsatility (for instance, the dynamical event may correspond to neural activity and/or an inflammatory response)

As describe above, the construction of conventional 2D ULM images is based on the (x, z) position of the ultrasound contrast agents or microbubbles signature on the image represented by the Point Spread Function (PSF). The local microbubble signature amplitude in the (x, z) plane is used to precisely locate the (x, z) position of that microbubble. Thus, conventional 2D ULM images are obtained by counting the number of microbubbles detected in each pixel, resulting in microbubbles count maps, commonly known as microbubbles density maps.

In case of the 1D array of ultrasonic transducers, from conventional ULM imaging, velocity maps may be computed based only on the interframe ultrasound contrast agent velocity vector components along the probe x-axis and the depth z-axis for conventional 2D imaging, due to the missing information along the direction Y.

Thus, the use of a 2D PSF function for a distribution of microbubbles in a 3D space does not allow the access to the information in the elevation direction, i.e in the direction along the axis Y, and in particular does not allow the location of microbubbles or ultrasound contrast agents flowing through the vessels crossing the XZ imaging plane.

One embodiment of the present disclosure is to provide a method that uses the amplitude of the isolated ultrasound agent backscattering signature which is a function of the (x, z) position of the microbubble in the 3D space, allowing the collection of the missing information in the elevation direction Y from ULM 2D imaging.

FIGS. 4(a)-4(c) depict the physical phenomenon linking the intensity backscattered by an ultrasound contrast agent or microbubble and its y position in the direction Y orthogonal to the imaging plane.

Each microbubble used in the ULM imaging method having a size smaller that the ultrasonic wavelength, it behaves as single Rayleigh scatter. In the case where the ultrasonic waves propagate in a homogenous medium and that all microbubbles have the same diameter and therefore the same cross section, the backscattered intensity from each microbubble depends only on the amplitude of the ultrasonic intensity received by the microbubble. Therefore, the amplitude of the signal from each microbubble is directly linked to its position in 3D space, within the transmitted ultrasound field. For a given position (x₀, z₀), as the ultrasonic intensity received by the microbubble varies along the axis Y as shown in FIG. 5(c), the ultrasound intensity backscattered by the microbubble varies along the axis Y. Consequently, determining the variation of the backscattering energy along the axis Y at a given depth Z allows the extraction of the information on the position of microbubbles in the elevation plane.

In FIG. 4(a), a microbubble 12 flows through a blood vessel 13 crossing the imaging plane 14, at two moments t₁ and t₂.

FIG. 4(b) shows that for the microbubble 12 located at the same position in the ULM image (x₀, z₀), but at two different positions y along the axis Y, due to the variation of the ultrasonic field along the axis Y, two different backscattered ultrasonic intensities are detected for the microbubble counted in that pixel.

FIG. 4(c) shows that for a given position (x₀, z₀) in the 2D ULM image, two backscattering intensities correspond to two position y₁, y₂ within the ultrasonic beam. The backscattering intensity corresponding to the position y₁ is lower than that of the position y₂. At t₂, because the microbubble is closer to the center of the ultrasound beam for the second position.

Therefore, backscattering amplitude imaging provides a new informative parameter linked to the position of the microbubbles in 3D space using a 1D array of transducers, within the ultrasound beam.

In an embodiment of the present disclosure, based on the filtered images obtained in step S3, a 2D backscattering amplitude ULM image may be computed S5 (B_ULM_COMP) by the computing module 3. During the step S3, isolated ultrasound agents are detected as brightest local maxima with a good correlation with the PSF on BMode images. The value of this maxima on the BMode images are extracted and associated with each localized ultrasound agent detection. This value is referred as the backscattering amplitude. Instead of attributing to each ULM image's pixel the number of microbubbles detected in this pixel as described in reference to step S4, the 2D backscattering amplitude image is constructed by attributing to each pixel a value representative of the measured backscattering amplitude of all ultrasound agent detected in that pixel. For instance, this value may be an average of the backscattering amplitudes of all ultrasound agents detected in that pixel. The grid chosen may be the same as the one chosen for the microbubbles count. Alternatively, this value may be a function of the backscattering amplitude of the at least one isolated ultrasound contrast agents detected in said pixel.

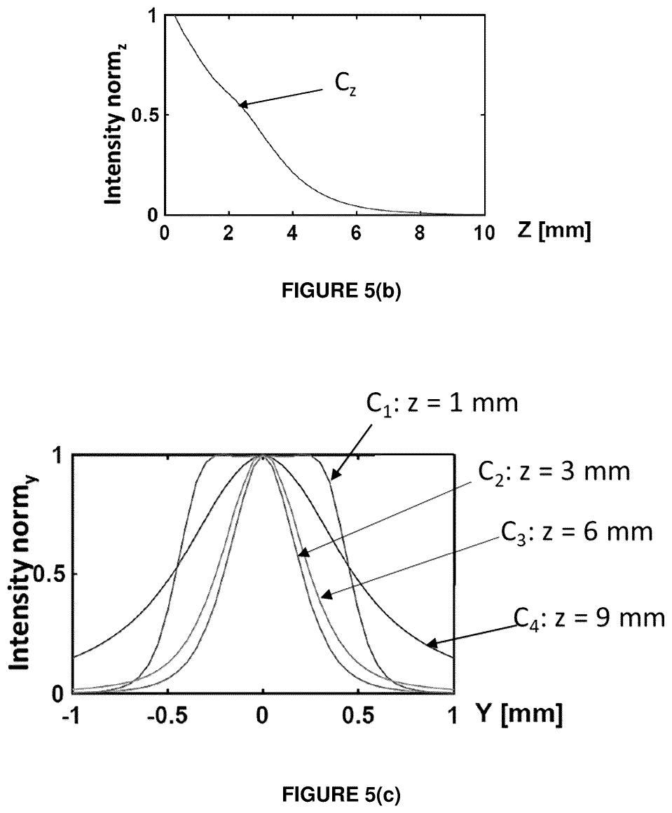

FIG. 6 shows a backscattering amplitude image obtained for a case of a vessel of a rat thalamus crossing the ultrasonic beam, i.e orthogonal to the imaging plane. The blood vessel extends in the out of plane and is represented by a white line on the image. On the backscattering amplitude image, such vessels crossing the imaging plane will appear the brightest where the vessels is in the center of the ultrasonic beam, gradually darkening as the vessels become closer to ultrasonic beam edges. In FIG. 6, for instance the arrows numbered 1, 3, 4 and 6 show the vessels located near the ultrasonic edges where the backscattering amplitude is low, the arrows numbered 2 and 5 show the vessels located at the center of the beam where the backscattering amplitude is maximal.

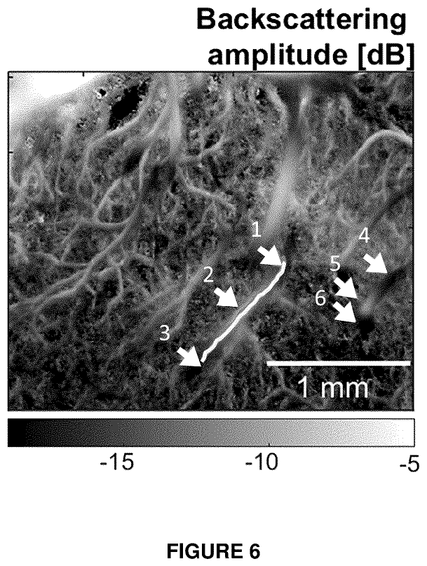

FIG. 7(a) illustrates the trajectory of a microbubble or an ultrasound contrast agent flowing through such vessel and its backscattering amplitude profile. As the microbubble enters the ultrasonic beam, the incident ultrasonic intensity it receives is minimal, therefore the backscattering intensity is minimal. As the microbubble arrives at the center of the ultrasonic beam where the incident intensity reaches a peak, the backscattered intensity is maximum. Then, the backscattering amplitude decreases as the microbubble leaves the ultrasonic beam.

FIG. 7(b) represents the normalized backscattering amplitude as a function of time detected for a microbubble flowing in the vessel whose trajectory is represented by a white line in FIG. 6.

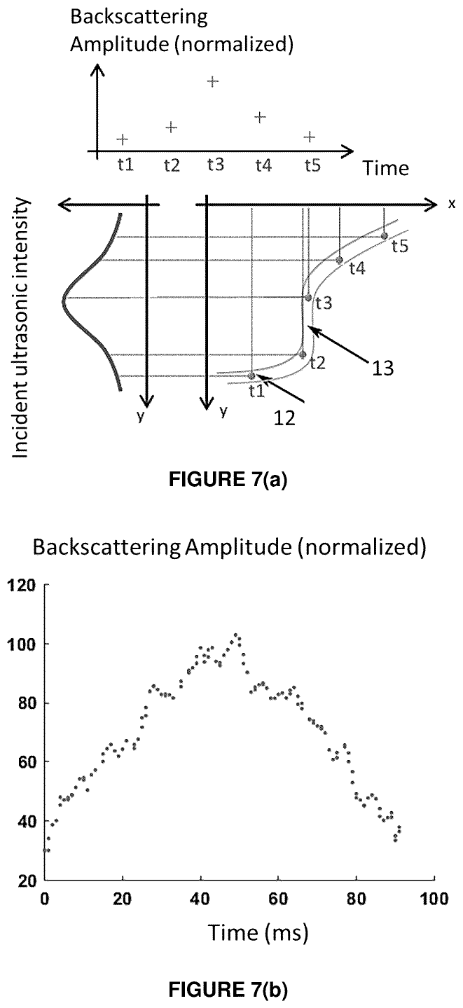

FIGS. 8(a) and 8(b) consider the case of vessels that are parallel to the imaging plane so that the backscattering amplitude from the microbubbles does not vary much. This situation corresponds to vessels belonging to the cortex for imaging a coronal or sagittal plane.

FIG. 8(a) represents schematically the backscattering intensity from the microbubbles 12 flowing in parallel vessels but located at different position along the axis Y. For instance, the vessel 15 is located at the center of the beam, where the ultrasonic intensity emitted by the linear array is maximal. The vessel 16 is located at the edge of the beam, where the ultrasonic field emitted by the linear array is minimal. Consequently, the intensity backscattered by the microbubbles flowing through the vessel 15 will be higher than that of microbubbles flowing through the vessel 16.

FIG. 8(b) shows three vessels of the cortex on a backscattering amplitude image. The vessel (arrow 3) closest to the middle of the beam are brighter and the vessels closer to the beam edges are darker (arrows 1 and 2).

In a case of a linear array of ultrasonic transducers, the measured backscattering amplitude of the microbubbles enable to retrieve the relative distance of the ultrasound contrast agents to the 2D imaging plane and the position y of the ultrasound contrast agents detected in the 2D ultrasound images. It is possible to calculate the three velocity components Vₓ, V_{y} and V_{z} along respectively the three directions X, Y, Z, improving accuracy of the speed computation of microbubbles. In other words, the present disclosure provides a method and apparatus to compute 3D flow and velocity from 2D ULM images obtained with a 1D ultrasound probe. The backscattering amplitude imaging permits to avoid the complex use of the 2D array of ultrasonic transducers.

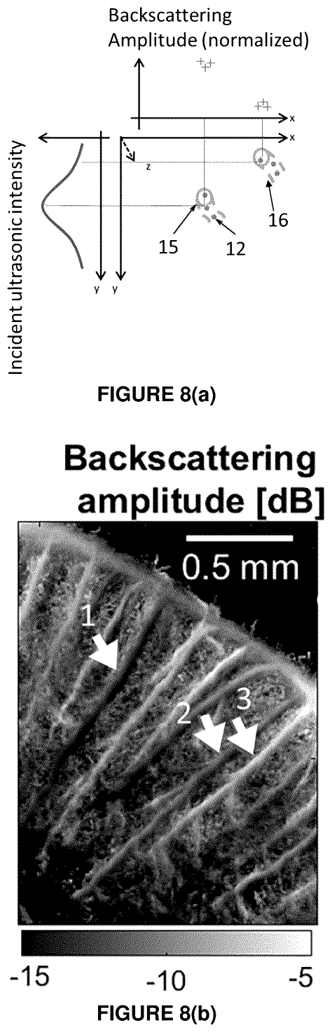

FIG. 9 illustrates how the localization of the ultrasound contrast agents in the out-of-plane direction is extracted from a series of 2D filtered images obtained with a plurality of parallel imaging planes.

Instead of using a single imaging plane to compute a single backscattering amplitude for a ultrasound contrast agent detected and tracked in the 2D filtered image, a spatial variation of backscattering amplitude for a ultrasound contrast agent at a given position (x, z) is generated as a function of y along the direction Y within the width w of the ultrasound beam and the position y of this isolated ultrasound contrast agent is computed by fitting a function on the variation of the backscattering amplitudes.

In an embodiment, the spatial variation of backscattering amplitude for the isolated ultrasound agent at a given position (x, z) may be generated as a function of the position y of the imaging plane in the direction Y within the width of the ultrasound beam. For instance, the processor 2 may be configured to move the 1D array 7 along the direction Y to generate a plurality of parallel ultrasound imaging planes spaced from each other within the width of the ultrasound beam in the direction Y. The spatial step between two successive imaging planes may be constant. The spatial step and the number of imaging planes are chosen so that the total length L of all imaging planes is equal or inferior to the width w of the ultrasonic beam. Thus, the same blood vessels are imaged. In other words, on all imaging planes, while the position of vessels in the plane XZ does not vary, the position along the axis Y may vary according to the planes. Thus, the ultrasonic intensity measured from the ultrasound contrast agent detected in each of 2D images associated with each of imaging planes for a given position (x, z) will vary.

In the example of FIG. 9, the probe is moved to generate 5 imaging planes along the Y axis with a 100 μm step between each of the 5 planes. As the beam width is over 500 μm, the same blood vessels are imaged by the ultrasound probe. For each imaging plane positioned at a given position y along the direction, a series of 2D filtered images is generated. Each series of filtered images is associated with a spatial position along the direction Y.

FIG. 9(a) illustrates this situation for three ultrasound contrast agent A, B, C. Each ultrasound contrast agent is imaged with five imaging planes plane 1, plane 2, plane 3, plane 4 and plane 5. Each ultrasound contrast agent does not receive the same incident ultrasound intensity for each imaging plane.

For the ultrasound contrast agent positioned in the pixel A, it is positioned outside of the ultrasound beam for the imaging planes 1, 2 and 3 and it is positioned at the beginning of the ultrasound beam for the imaging planes 4 and 5. Thus for the imaging planes 1, 2 and 3, the ultrasound contrast agent does not receive incident ultrasound intensity and there is no backscattered ultrasound intensity. For the imaging planes 4 and 5, the incident ultrasound intensity received by the microbubble and the backscattered ultrasound intensity is minimal. This spatial variation of the backscattered ultrasound intensity as a function of y is shown in FIG. 9(b).

For the ultrasound contrast agent positioned in the pixel B, it is positioned at the center of the ultrasound beam for the imaging plane 3. On the five images obtained, the intensity of the microbubble associated with the position (x_{B}, z_{B}) is maximal for the imaging plane 3 and will decrease towards the imaging plane 1 and towards the imaging plane 5. This variation of the backscattered ultrasound intensity as a function of y is shown in FIG. 9(a).

For the ultrasound contrast agent positioned in the pixel C, its situation is similar to the ultrasound contrast agent A. It is closer to the center of the ultrasound beam for the imaging plane 1 and moves away from the center towards the imaging plane 5. The backscattered ultrasound intensity decreases as a function of the position y of the imaging planes. This spatial variation of the backscattered ultrasound intensity as a function of y is shown in FIG. 9(b).

The spatial variation of the backscattering amplitude from the 5 imaging planes is used to compute the position y of the isolated contrast agent at a given position (x, z) in the direction Y by fitting a function on the variation of the backscattering amplitudes.

In an embodiment, the fitting function is a gaussian function:

$$a \times {e}^{- ( \frac{y - {y}_{0}}{w} ) 2} ,$$

a being the amplitude which decreases along the depth Z due to the ultrasonic attenuation, w being the width of the ultrasound beam which is larger near the transducers and convers towards the focal depth where it is minimal and then diverges, and y₀ being the position of the maximum of the variation of backscattering amplitudes and correspond to the position of the microbubble in the direction Y. The parameter w quantifying the width of the Gaussian fit may be an experimental value. Alternatively, the beam width may be obtained by simulation.

In the example of FIG. 9, five imaging planes are used to generate a curve comprising five backscattering amplitudes to perform the fit. The number of imaging planes and number of backscattering amplitudes may be adapted if necessary.

In the conventional 2D ULM imaging, the successive positions of ultrasound contrast agents in a track may be used to compute velocity parameters. In the example of FIG. 9, the velocity component along the probe X axis and the depth Z axis for the ultrasound contrast agent B at the position (x, z) may be computed. Thanks to the variation of the backscattering amplitude within the ultrasound beam width in the elevation direction, the sub-resolution position of the isolated microbubble is used to compute the velocity component along the Y axis. The absolute velocity magnitude may be computed. Thus, it is possible to compute accurately the velocity of the ultrasound contrast agent flowing in a blood vessel crossing the imaging plane.

In a similar manner, instead of determining the sub-resolution position of the isolated microbubble in the direction Y to compute the absolute velocity magnitude for each ultrasound contrast agent, it is possible to determine a variation of backscattering amplitude for each pixel at a given position (x, z) as a function of the position y along the direction Y and the position y of each pixel (x, z) in the direction Y is computed by fitting a function on the variation of the backscattering amplitudes.

The computation speed step for each pixel at a given position (x, z) comprises the following steps:
(i) imaging the area of interest for a plurality of parallel ultrasound imaging planes XZ spaced from each other within the width of the ultrasound beam along the direction Y;
(ii) generating a 2D backscattering amplitude image for each imaging plane XZ by attributing for each pixel a value representative of the measured backscattering amplitude of at least one isolated ultrasound contrast agent detected in said pixel;
(iii) generating a spatial variation of the backscattering amplitude for each pixel as a function of the position y of the imaging planes along the direction Y within the width of the ultrasound beam;
(iv) computing the position y of each pixel by fitting a function on the spatial variation of the measured backscattering amplitude, the position y of each pixel being the position of the maximum of the variation of backscattering amplitudes;
(v) computing three velocity components Vₓ, V_{y} and V_{z} along respectively the three directions X, Y, Z for each pixel.

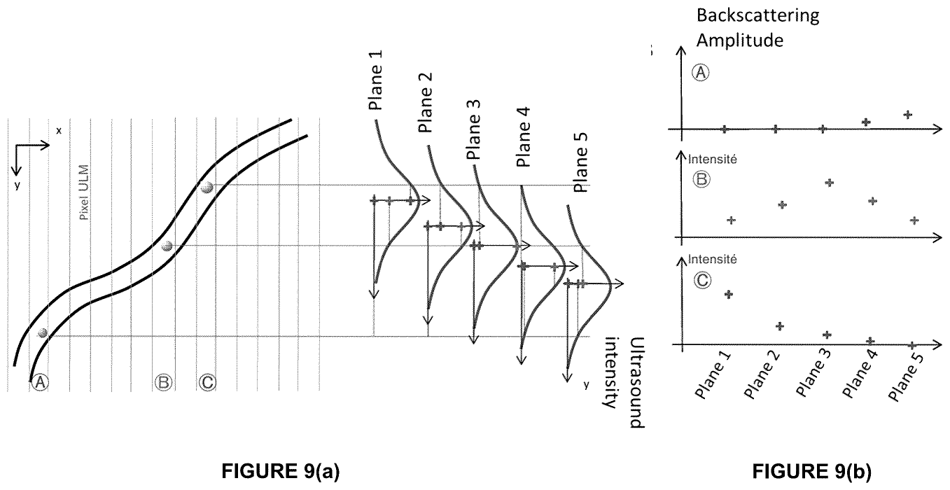

FIG. 10 illustrates five backscattering amplitude images associated with five parallel imaging planes positioned along the direction Y withing the beam width. A variation of backscattering amplitude for a pixel M at a given position (x, z) is generated as a function of y along the direction Y within the width w of the ultrasound beam. Then, the position y of this pixel is computed by fitting a function on the variation of the backscattering amplitudes.

Five backscattering amplitude images are generated for five coronal imaging planes spaced by 100 μm from each other in the elevation Y direction in a rat thalamus. Thus, the five imaging planes are positioned within the width of the ultrasound beam. The same blood vessels are present on the five images shown in FIG. 9. For each pixel of the five images at a given position (x, z), a variation of backscattering amplitude is generated as a function of the position of the imaging plane along the direction Y within the width w of the ultrasound beam. For each pixel (x, z), a function may be fitted on the curve backscattering amplitudes measured for the five imaging planes. This fit is used to determine the sub-resolution position of the maximum of the backscattering amplitude function, corresponding to the position y₀ of the pixel in the elevation direction. For instance, the fit may be a Gaussian fit:

$$a \times {e}^{- ( \frac{y - {y}_{0}}{w} ) 2} ,$$

a being the amplitude which decreases along the depth Z due to the ultrasonic attenuation, w being the width of the ultrasound beam which is larger near the transducers and convers towards the focal depth where it is minimal and then diverges, and y₀ being the position of the maximum of the variation of backscattering amplitudes and correspond to the position of the pixel in the direction Y. The parameter w quantifying the width of the Gaussian fit may be an experimental value. Alternatively, the beam width may be obtained by simulation.

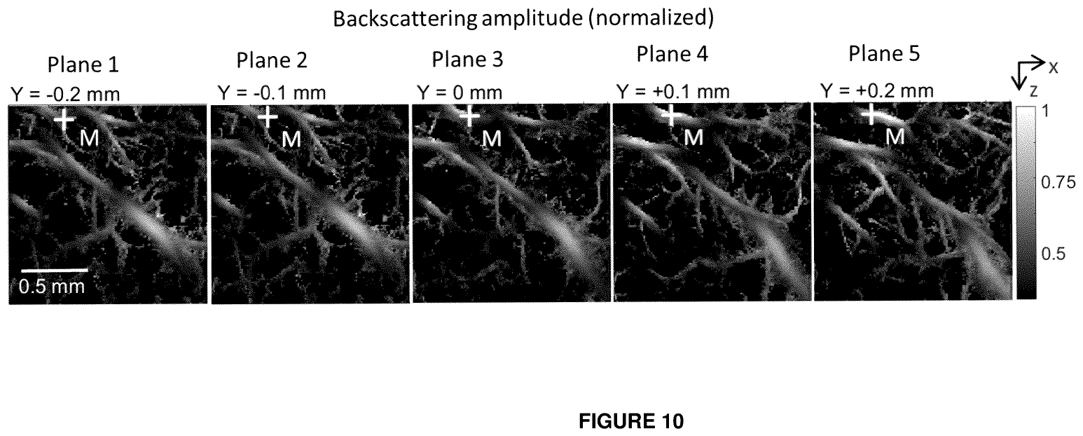

FIG. 11 illustrates five backscattering amplitudes measure for the pixel M represent by a cross in FIG. 10. The curve G is a Gaussian fit on the 5 backscattering amplitudes. The position of this pixel M is given by the y position where the fit is maximum: y₀. In the present example, the y₀=20 μm.

It is possible to localize each pixel (x, z) in the direction Y using the backscattering information available in 2D ULM imaging in the plane XZ. It also possible to compute the velocity component along the probe X axis and the depth Z axis for each pixel at the position (x, z) and the velocity component along the Y axis. The absolute velocity magnitude may be computed for each pixel. Thus, it is possible to compute accurately the velocity of the blood vessel crossing the imaging planes and generate a 3D velocity map.

In another embodiment, the computing module 3 is adapted to correct the positioning of the 1D array of ultrasonic transducers along the direction Y within the width of the ultrasound beam by comparing the backscattering amplitude images generated from two set of raw data acquired at two different moments. For instance, a first set of raw data is acquired by the 1D array positioned at a given position y₁ along the axis Y with a first imaging plane. A first backscattering amplitude image is generated from this first set of raw data as explained above, by attributing for each pixel a parameter related to the backscattering amplitude for all microbubbles or ultrasound contrast agents detected in this pixel. A second set of raw data is acquired by the same 1D array. A second backscattering amplitude image is generated from this second set of raw data. In case where it is necessary to position the 1D array to image an area with the same image imaging plane as the first measurement configuration, the computing module 3 is configured to compare the first backscattering amplitude image and the second backscattering for each pixel to determine if the imaging plane associated to the first backscattering amplitude image coincides with the imaging plane associated to the second backscattering image.

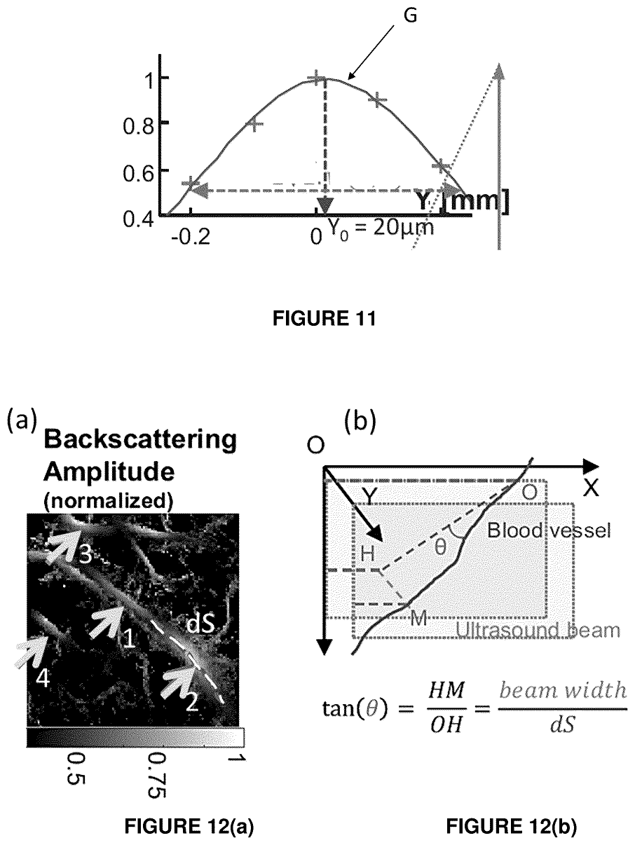

FIG. 12 illustrates how the missing component of the velocity in the direction Y may be retrieved from the backscattering amplitude image generated with a single imaging plane acquisition (x, z).

In the convention ULM imaging, the velocity of the blood can be only computed from the two in-plane components of the velocity vector, i.e. the components Vₓ and V_{z}. The in plane 2D components of the blood velocity is determined using the differential of the successive positions of ultrasound contrast agents in a track. Consequently, the velocity is underestimated, especially for blood vessels crossing the imaging planes.

The backscattering information from a single plane acquisition is used to determine the position of the blood vessel relative to imaging plane. In addition, the method comprises a quantification step of the corrected velocity of at least one vessel from a backscattering amplitude image acquired with a single imaging plane. This quantification step uses a geometric modelling of the out-of-plane angle of the vessel to deduce the missing component, by assuming a rectilinear crossing of the ultrasound beam by the vessel as illustrated in FIG. 12(b).

The quantification step comprises the following steps:
(i) selecting at least one blood vessel in the at least one backscattering amplitude image generated in the step S5. For instance, FIG. 12 shows four selected blood vessel 1-4;
(ii) computing the in plane velocity V_{xz} of the ultrasound contrast agents belonging to said selected blood vessel as described above;
(iii) obtaining the backscattering amplitude profile along the centerline of said selected blood vessel;
(iii) computing the angle θ as tan (0)=HM/OH, e being the angle between the backscattering amplitude profile along the selected blood vessel centerline and its projection on the ultrasound imaging plane. HM is the beam width and OH is dS which may be evaluated by the Full Width Half Maximum (FWHM) of the backscattering profile along the curvilinear abscissa following the centerline as shown in FIG. 12(a);
(iv) computing a corrected velocity for each ultrasound contrast agents belonging to said selected vessel using said computed angle: V_{corr}=V_{xz}/cos θ.

Owing to the measurement of the backscattering amplitudes in ULM imaging, the computing module 3 is adapted to delineate more small vessels than conventional ULM images due to an intrinsic lower spatial noise. Backscattering ULM imaging is less sensitive to noise, allowing the detection of small vessels compared to conventional ULM images which are base d on the ultrasound contrast agents count. In conventional ULM images, each pixel is associated with the ultrasound contrast agents count. Thus, the signal is driven by the blood flow. In small vessel, as the blood flow is lower, the signal is lower, rendering its detection difficult. In backscattering imaging, the signal is driven by the position of the blood vessels within the ultrasound beam independently from hemodynamics. Although the backscattering amplitude image and the conventional ULM images are generated from the same set of raw data measures by the 1D array of transducers, the noise signal has a lower influence in backscattering imaging than in conventional ULM.

Advantageously, backscattering imaging may be more adapted for segmentation algorithms and may provide better insights on the size and order of branching capillaries detected than using ULM imaging.

In one embodiment, the method comprises an automatic segmentation step of the vessels in at least one area of a vascular network from a 2D backscattering amplitude image or a 3D backscattering amplitude image. Thus, it is possible to quantify at least one dimensional parameter of the vessels from the segmented image. For example, the dimensional parameter may be the vessels diameter or the length of each segment of vessels.

In addition, the backscattering amplitude ULM imaging allows more accurate speed quantifications. Owen the multi-imaging planes, the localization technique combined with backscattering amplitude imaging provides the 3D localization of the blood vessels with a sub-wavelength resolution in the out-of-plane direction. Using one single imaging plane acquisition, although the exact location of the ultrasound contrast agents relative to the imaging plane cannot be retrieved, it is possible to estimate the angle between the blood vessel and its projection on the imaging plane to provide a 3D correction of the speed vector.

Based on the measured backscattering amplitudes for each isolated contrast agents, and from the backscattering amplitude images, at least one vascular dynamic parameter may be computed accurately. The at least one vascular dynamic parameter may be chosen in the group comprising: blood flow, blood velocity, blood volume, blood pressure, vascular vessels' diameters, and any combination thereof.

In the present disclosure, the vascular network described is the vascular network of a nervous system. Nevertheless, the methods and apparatus described may be adapted to any vascular network.

1. A method for imaging vascular activity at a microscopic scale in at least one area of a vascular network of an organ, of a human or animal, the method including:
(a) transmitting a series of successive incident ultrasonic waves in the at least one area by an array of ultrasonic transducers, the array of ultrasonic transducers extending along at least one direction and the incident ultrasonic waves being propagated in a direction perpendicular to the array of transducers;
(b) acquiring a set of raw data from backscattered ultrasonic waves by said array of transducers;
(c) generating a series of successive ultrasound images from said raw data;
(d) detecting at least one isolated ultrasound contrast agent in the ultrasound images;
(e) localizing the position of said at least one isolated ultrasound contrast agent with a precision inferior to the wavelength of the waves; and
(f) generating an at least 2D backscattering amplitude image by attributing for each pixel a value representative of the measured backscattering amplitude of at least one isolated ultrasound contrast agent detected in said pixel.

2. The method according to claim 1, wherein the value computed in step (f) corresponds to an average of the measured backscattering amplitude of all isolated ultrasound contrast agents detected in said pixel.

3. The method according to claim 1, wherein the value computed in step (f) corresponds to a function of the backscattering amplitude of the at least one isolated ultrasound contrast agents detected in said pixel.

4. The method according to claim 1, further comprising a tracking step wherein at least one isolated ultrasound contrast agent detected is tracked across several ultrasound images generated in step (c) to determine its trajectory and its speed.

5. The method according to claim 1, wherein the array of ultrasonic transducers is a 1D array extending along one direction X and the incident ultrasonic waves being propagated in a direction Z perpendicular to the array of transducers, said image generated in step (f) is a 2D backscattering amplitude image.

6. The method according to claim 5, wherein the steps (a) to (f) are repeated for a plurality of parallel ultrasound imaging planes XZ spaced from each other within the width of the ultrasound beam along the direction Y to generate a plurality of 2D parallel backscattering amplitude images.

7. The method according to claim 4, further comprising a computation speed step for the at least one tracked isolated ultrasound agent at a given position (x, z) comprising the following steps:
(i) generating a spatial variation of the backscattering amplitude for said at least one tracked isolated contrast agent as a function of the position y of the imaging plane along the direction Y within the width of the ultrasound beam;
(ii) computing the position y of said at least one tracked isolated contrast agent by fitting a function on the spatial variation of the measured backscattering amplitude, the position y of the at least one isolated contrast agent being the position of the maximum of the spatial variation of backscattering amplitudes; and
(iii) computing the velocity components Vₓ, V_{y} and V_{z} along respectively the three directions X, Y, Z for said at least one tracked isolated ultrasound contrast agent.

8. The method according to claim 6, further comprising a computation speed step for each pixel at a given position (x, z) comprising the following steps:
(i) generating a spatial variation of the backscattering amplitude for each pixel as a function of the position y of the imaging planes along the direction Y within the width of the ultrasound beam;
(ii) computing the position y of each pixel by fitting a function on the spatial variation of the measured backscattering amplitude, the position y of each pixel being the position of the maximum of the variation of backscattering amplitudes; and
(iii) computing three velocity components Vₓ, V_{y} and V_{z} along respectively the three directions X, Y, Z for each pixel.

9. The method according to claim 7, wherein the fitting function is a Gaussian function:
$$a \times {e}^{- ( \frac{y - {y}_{0}}{w} ) 2} ,$$
a being the backscattering amplitude, w being the width of the ultrasound beam along the direction Y, and y₀ being the position of the maximum of the variation of backscattering amplitudes.

10. The method according to claim 9, wherein the width w of the ultrasound beam along the direction Y is an experimental value.

11. The method according to claim 9, wherein the width w of the ultrasound beam along the direction Y is a simulated value.

12. The method according to claim 6, wherein the plurality of parallel ultrasound imaging planes are obtained by moving the 1D array of ultrasonic transducers along a direction Y perpendicular to the ultrasound imaging plane (x, z) within the width (w) of the ultrasound beam.

13. The method according to claim 6, further comprising a correction step of the positioning of the 1D array of ultrasonic transducers by comparing a first backscattering amplitude image and a second backscattering so as the imaging plane associated to the first backscattering amplitude image coincides with the imaging plane associated to the second backscattering image.

14. The method according to claim 1, further comprising a step of generating an at least 2D ultrasound localization microscopy (ULM) image of the at least one area is generated by attributing for each pixel a number of ultrasound contrast agents detected in said pixel.

15. The method according to claim 5, further comprising a quantification step of the corrected velocity for at least one vessel from said 2D backscattering amplitude image, said quantification step comprising:
(i) selecting said at least one blood vessel in the at least one backscattering amplitude image;
(ii) computing the velocity V_{xz} of the ultrasound contrast agents belonging to said selected blood vessel;
(ii) obtaining the backscattering amplitude profile along a centerline of said selected blood vessel;
(iii) computing the angle θ between the backscattering amplitude profile along the selected blood vessel centerline and its projection on the ultrasound imaging plane; and
(iv) computing a corrected velocity for each ultrasound contrast agents belonging to said selected vessel using said computed angle: V_{corr}=V_{xz}/cos θ.

16. The method according to claim 1, wherein the step of generating a series of successive 2D ultrasound images from said raw data comprises a step of filtration to discriminate the ultrasound signal of the individual ultrasound contrast agents from a tissue signal.

17. The method according to claim 1, wherein at least one vascular parameter is extracted from the measured backscattering amplitudes, said one vascular parameter being chosen in the group comprising: blood flow, blood velocity, blood volume, blood pressure and any combination thereof.

18. The method according to claim 1, further comprising an automatic segmentation step of the vessels in at least one area of a vascular network from the at least 2D backscattering amplitude image and wherein at least one dimensional parameter of the vessels is quantified from the segmentation step.

19. An apparatus for imaging vascular activity at a microscopic scale in at least one area of a vascular network of an organ, of a human or animal, said apparatus including:
(a) an ultrasound probe having an array of ultrasonic transducers extending along at least one direction, said ultrasound probe configured to transmit incident ultrasound waves in a direction of propagation perpendicular to the array of transducers and acquire a set of raw data;
(b) a computing module configured to:
generate a series of successive ultrasound images from said raw data;
detect at least one isolated ultrasound contrast agent in the ultrasound images;
localize the position of said at least one isolated ultrasound contrast agent with a precision inferior to the wavelength of the waves;
track said at least one isolated ultrasound contrast agent to determine its trajectory; and
generate an at least 2D backscattering amplitude image by attributing for each pixel a value representative of the measured backscattering amplitude of at least one isolated ultrasound contrast agent detected in said pixel.

20. The apparatus according to claim 19, wherein the array of ultrasonic transducers is 1D array of ultrasonic transducers extending along one direction X to generate 2D backscattering amplitude images.

21. The apparatus according to claim 20, wherein the probe further comprises a motor configured to move the array of ultrasonic transducers along a direction perpendicular to the ultrasound imaging plane within the width (w) of the ultrasound beam to generate a plurality of parallel ultrasound imaging planes.

22. The apparatus according to claim 21, wherein the computing module is further configured to:
generate a spatial variation of the backscattering amplitude for said at least one tracked isolated contrast agent as a function of the position y of the imaging planes along the direction Y within the width of the ultrasound beam;
compute the position y of said at least one tracked isolated contrast agent by fitting a function on the spatial variation of the measured backscattering amplitude, the position y of the at least one isolated contrast agent being the position of the maximum of the variation of backscattering amplitudes; and
compute three velocity components Vₓ, V_{y} and V_{z} along respectively the three directions X, Y, Z for said at least one tracked isolated ultrasound contrast agent.

23. Computer software comprising instructions to implement a method for imaging vascular activity at a microscopic scale in at least one area of a vascular network of an organ, of a human or animal, when the software is executed by a processor, said method including:
(a) transmitting a series of successive incident ultrasonic waves in the at least one area by an array of ultrasonic transducers, the array of ultrasonic transducers extending along at least one direction and the incident ultrasonic waves being propagated in a direction perpendicular to the array of transducers;
(b) acquiring a set of raw data from backscattered ultrasonic waves by said array of transducers;
(c) generating a series of successive ultrasound images from said raw data;
(d) detecting at least one isolated ultrasound contrast agent in the ultrasound images;
(e) localizing the position of said at least one isolated ultrasound contrast agent with a precision inferior to the wavelength of the waves; and
(f) generating an at least 2D backscattering amplitude image by attributing for each pixel a value representative of the measured backscattering amplitude of at least one isolated ultrasound contrast agent detected in said pixel.

24. Computer-readable non-transient recording medium on which a software is registered to implement a method for imaging vascular activity at a microscopic scale in at least one area of a vascular network of an organ, of a human or animal, when the software is executed by a processor, said method including:
(a) transmitting a series of successive incident ultrasonic waves in the at least one area by an array of ultrasonic transducers, the array of ultrasonic transducers extending along at least one direction and the incident ultrasonic waves being propagated in a direction perpendicular to the array of transducers;
(b) acquiring a set of raw data from backscattered ultrasonic waves by said array of transducers;
(c) generating a series of successive ultrasound images from said raw data;
(d) detecting at least one isolated ultrasound contrast agent in the ultrasound images;
(e) localizing the position of said at least one isolated ultrasound contrast agent with a precision inferior to the wavelength of the waves; and
(f) generating an at least 2D backscattering amplitude image by attributing for each pixel a value representative of the measured backscattering amplitude of at least one isolated ultrasound contrast agent detected in said pixel.
